(19) United States
(12) Patent Application Publication
(10) Pub. No.: US 2023/0248507 A1
(43) Pub. Date: Aug. 10, 2023

(54) NOA - Nitinol Orthodontic Appliance

(71) Applicant: Myles Egbowon, Naperville, IL (US)

(72) Inventor: Myles Egbowon Egbowon, Chicago, IL (US)

(21) Appl. No.: 17/666,356

(22) Filed: Feb. 7, 2022

(51) Int. Cl. A61F 2/08; C22F 1/00

(52) U.S. Cl. CPC A61F 2/08; C22F 1/006

(57) ABSTRACT

The present invention uses nitinol to augment and fully develop the maxilla, using an ambient temperature powered system. This device fixes facial deformities, while fully developing the face. This device is mainly used for its cosmetic and airway benefits, but not limited in application. Not only does it move the maxilla, but it moves the mandible, through facial cranial development. This “second” tongue is extremely strong, allowing for results that would not be available in nature.

### BACKGROUND

The present invention relates to the correction of facial structure development caused frequently by partial or complete reduction of maxillary pressure. Often mouth breathing allows the tongue to be displaced posteriorly and tends to partially or completely deform the face. The expansion or widening of the maxilla can significantly improve facial appearance, which can also contribute to the correction of mouth breathing.

### SUMMARY

Nitinol is used to create an orthodontic appliance that moves the maxilla in an extreme and high pressure fashion. With the right metrics, the device can upturn the maxilla in a matter of days, due to the ambient temperature of the body powering the overwhelming strength of the device. This in turn, makes the ace extremely attractive, allowing the user to achieve their maximum potential in regards to Facial structure. Think of this device as an enchantment to the body, allowing for an extremely good looking appearance.

### BRIEF DESCRIPTION OF THE DRAWINGS

Various embodiments of the invention are disclosed in the following detailed description and accompanying drawings.

FIG. 1 illustrates a nitinol device with molar attachments, and the system of ravelment; Shows the device raveled and unraveled.

### DETAILED DESCRIPTION AND PREFERRED EMBODIMENT

Using nitinol, we can create a medical device that allows for the maxillary expansion of the face, laterally, and width wise. Nitinol is used as a curved rectangular piece in the mouth, and curls up next to the maxilla and into a fully formed maxilla. The strength of nitinol allows for the hyperexpansion of the maxilla. The device works on curved tension on the maxilla. This device works by mimicking the tongue in shape and action, tuning the nitinol into a 37 degree celsius temperature. Therefore, the device runs on the ambient temperature of the body. In comparison, the human tongue has a tensile strength of 63 kpa. The nitinol tongue has a tensile strength of 100-1,000 MPa, or 100,000 kpa. This allows for rapid maxillary expansion and development, leading to a full development of the face. Facial changes can be achieved in a matter of hours of use. The device is positioned in a way that focuses the tip of the device at the top of the roof of the mouth. The device uses a dental adhesive on the side acing the maxilla to stick the nitinol on the top of the mouth securely.

### Definitions

A. Nitinol
a. Nickel titanium, also known as Nitinol, is a metal alloy of nickel and titanium, where the two elements are present in roughly equal atomic percentages. Different alloys are named according to the weight percentage of nickel; e.g., Nitinol 55 and Nitinol 60. It exhibits the shape memory effect and superelasticity at different temperatures.

Various embodiments of the invention are disclosed in the following detailed description and accompanying drawings.

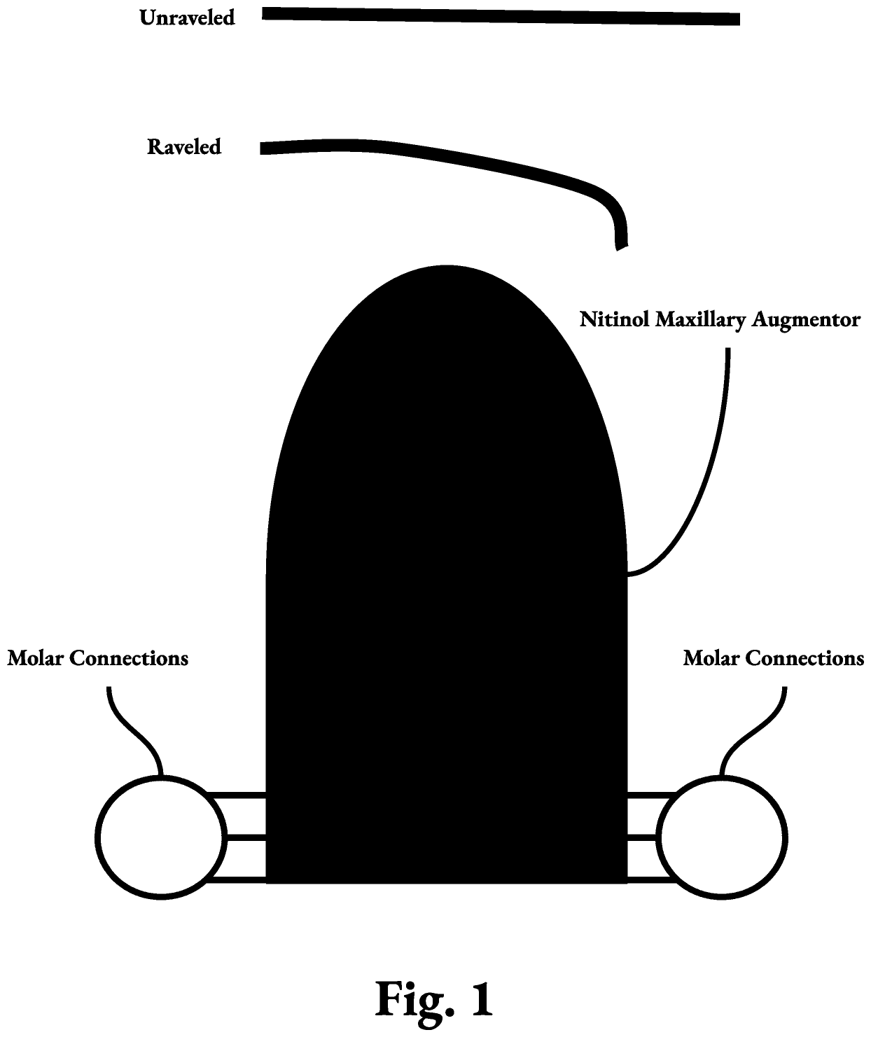

FIG. 1 illustrates a nitinol device with molar attachments, and the system of ravelment; Shows the device raveled and unraveled.

### Detailed Description and Preferred Embodiment

Using nitinol, we can create a medical device that allows for the maxillary expansion of the face, laterally, and width wise. Nitinol is used as a curved rectangular piece in the mouth, and curls up next to the maxilla and into a fully formed maxilla. The strength of nitinol allows for the hyperexpansion of the maxilla. The device works on curved tension on the maxilla. This device works by mimicking the tongue in shape and action, tuning the nitinol into a 37 degree celsius temperature. Therefore, the device runs on the ambient temperature of the body. In comparison, the human tongue has a tensile strength of 63 kpa. The nitinol tongue has a tensile strength of 100-1,000 MPa, or 100,000 kpa. This allows for rapid maxillary expansion and development, leading to a full development of the face. Facial changes can be achieved in a matter of hours of use. The device is positioned in a way that focuses the tip of the device at the top of the roof of the mouth. The device uses a dental adhesive on the side facing the maxilla to stick the nitinol on the top of the mouth securely.

### Definitions

A. Nitinol
a. Nickel titanium, also known as Nitinol, is a metal alloy of nickel and titanium, where the two elements are present in roughly equal atomic percentages. Different alloys are named according to the weight percentage of nickel; e.g., Nitinol 55 and Nitinol 60. It exhibits the shape memory effect and superelasticity at different temperatures.

### CONCLUSION

The disclosed embodiments are illustrative, not restrictive. While specific configurations of the gene therapy have been described, it is understood that the present invention can be applied to a wide variety of orthodontics. There are many alternative ways of implementing the invention.

What is claimed is:

1. The use of nitinol to change the shape of the maxilla with a medical device,
a. The use of curved tension on the maxilla to change its shape with a medical device.
